(19) United States
(12) Patent Application Publication
(10) Pub. No.: US 2025/0276196 A1
(43) Pub. Date: Sep. 4, 2025

(54) TREATMENT APPARATUS AND METHODS FOR TREATMENT OF WOUNDS

(71) Applicant: Danvantar Biophotonics Oy, Helsinki (FI)

(72) Inventor: Matti Juhani Myllymäki, Helsinki (FI)

(73) Assignee: Danvantar Biophotonics Oy, Helsinki (FI)

(21) Appl. No.: 18/592,865

(22) Filed: Mar. 1, 2024

(51) Int. Cl. A61N 5/06

(52) U.S. Cl. CPC A61N 5/0624; A61N 5/0616; A61N 2005/0628; A61N 2005/0629; A61N 2005/0651; A61N 2005/0659; A61N 2005/0661; A61N 2005/0663

(57) ABSTRACT

Disclosed is a treatment apparatus (100) for treatment of a wound (102). The treatment apparatus comprises: a processor (104) and a light source (106) arranged to emit light (108) towards the wound when in use. The processor is configured to control the light source to emit: during a first 5period (202) of time, a light comprising a first plurality of wavelength ranges, wherein at least one of the first plurality of wavelength ranges is a first disinfecting wavelength range of light having a first radiant power; and during a second period (204) of time, a light comprising a second plurality of wavelength ranges, wherein at least one of the second plurality of wavelength ranges is the first disinfecting range having the first radiant power or a second disinfecting wavelength range of light having a second radiant power.

### TECHNICAL FIELD

The present disclosure relates to treatment apparatuses for treatment of wounds. The present disclosure also relates to methods of treatment of chronic wounds of a first type. The present disclosure also relates to methods of treatment of chronic wounds of a second type.

### BACKGROUND

Wounds and skin diseases are prevalent health issues affecting millions of people worldwide. According to existing knowledge, healing of such wounds and skin diseases is achieved through the interaction of three key components: a group of precursor cells that can proliferate and differentiate into fibroblasts and keratinocytes, neo-angiogenesis to restore blood circulation to a site of injury or disease and to deliver nutrients and cells to the wound or skin disease, and a balanced immune system capable of eliciting a controlled inflammatory response.

However, when said interaction is disrupted, healing stops and this can result in chronic, non-healing inflammation. Chronic wounds and skin diseases are characterized by a stagnation or, in autoimmune cases, an overactivation during a progression of exacerbation phases, especially during an inflammatory phase. Infections are also a common trigger for development of chronic inflammations and complicate the healing of various wounds and skin diseases. Since skin and wounds are non-sterile environments, infections occur across a spectrum ranging from contamination and colonization to localized infection and spreading infection to systemic infection.

In recent years, light therapy (namely, phototherapy) has emerged as a promising treatment for various skin diseases and wounds. Light therapy involves exposure to specific wavelengths of light, for specific periods of time, to stimulate biological processes in skin. However, a major challenge associated with light therapy is that there do not exist precise prescriptions related to the specific wavelengths of light and specific periods of time, for treating different wound types or skin infections. Determining a right combination for a recipient of light therapy for a target wound type requires considerable experimentation, which is not always feasible.

Furthermore, existing apparatuses for light therapy are typically configured to provide light over only a single range of wavelengths. In practical terms, there is a very large number of feasible permutations and of wavelengths and treatment times which can be used for light therapy. As an example, one can select one or more wavelength ranges, provide those with certain radiant power, for a certain time in a certain order, for treatment of different wound types. The existing apparatuses for light therapy thus have very limited applicability, and programming them for light therapy requires substantial work.

Therefore, in light of the foregoing discussion, there exists a need to overcome the aforementioned drawbacks.

### SUMMARY

The present disclosure seeks to provide a treatment apparatus and methods for treatment of wounds. The aim of the present disclosure is achieved by a treatment apparatus and methods which incorporate emission of light of different wavelengths to treat the wound, as defined in the appended independent claims to which reference is made to. Advantageous features are set out in the appended dependent claims.

Throughout the description and claims of this specification, the words “comprise”, “include”, “have”, and “contain” and variations of these words, for example “comprising” and “comprises”, mean “including but not limited to”, and do not exclude other components, items, integers or steps not explicitly disclosed also to be present. Moreover, the singular encompasses the plural unless the context otherwise requires. In particular, where the indefinite article is used, the specification is to be understood as contemplating plurality as well as singularity, unless the context requires otherwise.

### BRIEF DESCRIPTION OF THE DRAWINGS

FIG. 1 is a schematic illustration of a treatment apparatus for treatment of a wound, in accordance with an embodiment of the present disclosure;

FIG. 2 is an exemplary timeline of treatment of a wound, in accordance with an embodiment of the present disclosure;

FIG. 3 is a graphical illustration of an exemplary spectral profile of a given wavelength range, in accordance with an embodiment of the present disclosure;

FIG. 4 is an exemplary contour plot for treatment of a wound, in accordance with an embodiment of the present disclosure;

FIG. 5 illustrates steps of a method of treatment of a chronic wound of a first type, in accordance with an embodiment of the present disclosure;

FIG. 6 illustrates steps of a method of treatment of a chronic wound of a second type, in accordance with an embodiment of the present disclosure; and

FIG. 7 is an example spectrum measured from an apparatus in accordance with an embodiment of the present disclosure.

### DETAILED DESCRIPTION OF EMBODIMENTS

The following detailed description illustrates embodiments of the present disclosure and ways in which they can be implemented. Although some modes of carrying out the present disclosure have been disclosed, those skilled in the art would recognize that other embodiments for carrying out or practising the present disclosure are also possible.

In a first aspect, an embodiment of the present disclosure provides a treatment apparatus for treatment of a wound, the treatment apparatus comprising:
a processor and a light source, wherein the light source is arranged to emit light towards the wound when in use and
wherein the processor is configured to control the light source to emit:
during a first period of time, a light comprising a first plurality of wavelength ranges, wherein at least one of the first plurality of wavelength ranges is a first disinfecting wavelength range of light having a first radiant power; and
during a second period of time, a light comprising a second plurality of wavelength ranges, wherein at least one of the second plurality of wavelength ranges is the first disinfecting range having the first radiant power or a second disinfecting wavelength range of light having a second radiant power.

The aforementioned first aspect provides the treatment apparatus for diverse therapeutic treatments, by constructing a recipe built from various wavelengths of light, wherein each wavelength of light exerts a unique impact. This contrasts with traditional recipes that focus on a few healing wavelengths targeting a limited scope of healing factors. Analogous to pharmaceuticals used in wound care, traditional treatments often rely on broad-spectrum antibiotics, whereas the processor is configured to emit light which has wide-ranging disinfectant (antibiotic) effective against all pathogens, including those resistant to conventional antibiotics and antifungal medications. Moreover, traditional treatments lack optimal timing and dosage due to the need for multiple devices and simultaneous wavelengths, which are not balanced in their timing and doses. However, the processor is configured control the light source to emit the light during the first period of time and the second period of time, which is quite balanced in their timing. Moreover, the processing steps can be implemented with ease. In this way, the treatment apparatus facilitate a user-friendly, simple and effective way to treat the wound.

In a second aspect, an embodiment of the present disclosure provides a method of treatment of a chronic wound of a first type, wherein the wound is treated by providing during:
a first period of time of 30 to 90 seconds, a first plurality of wavelength ranges and radiant powers of:
   a first disinfecting wavelength range is from 200 nm to 280 nm and a first radiant power is between 50 uW/cm² to 1mW/cm²,
   a second disinfecting wavelength range is from 300 nm to 390 nm and a second radiant power is between 100 uW/cm² to 25 mW/cm², and
   a third disinfecting wavelength range is from 400 nm to 430 nm and a third radiant power is between 3 mW/cm² to 200 mW/cm²,
   and
a second period of time of 60 to 180 seconds, a second plurality of wavelength ranges and radiant powers of:
   the first disinfecting wavelength range is from 200 nm to 280 nm and the first radiant power is between 50 uW/cm² to 1 mW/cm²,
   a fourth disinfecting wavelength range is from 610 nm to 660 nm and a fourth radiant power is between 0.5 mW/cm² to 200 mW/cm², and
   a fifth wavelength range is from 820 nm to 900 nm and a fifth radiant power is between 1.5 mW/cm² to 200 mW/cm²,
wherein each respective radiant power is a radiant power of respective wavelength range of light at the wound.

In a third aspect, an embodiment of the present disclosure provides a method of treatment of a chronic wound of a second type, wherein the wound is treated by providing during:
a first period of time of 30 to 90 seconds, a first plurality of wavelength ranges and radiant powers of:
   a first disinfecting wavelength range is from 200 nm to 280 nm and a first radiant power is between 50 uW/cm² to 1 mW/cm²,
   a fourth disinfecting wavelength range is from 610 nm to 660 nm and a fourth radiant power is between 0.5 mW/cm² to 200 mW/cm², and
   a fifth wavelength range is from 820 nm to 900 nm and a fifth radiant power is between 1.5 mW/cm² to 200 mW/cm²,
a second period of time of 30 to 90 seconds, a second plurality of wavelength ranges and radiant powers of:
   a second disinfecting wavelength range is from 300 nm to 390 nm and a second radiant power is between 100 uW/cm² to 25 mW/cm²,
   the fourth disinfecting wavelength range is from 610 nm to 660 nm and the fourth radiant power is between 0.5 mW/cm² to 200 mW/cm², and
   the fifth wavelength range is from 820 nm to 900 nm and the fifth radiant power is between 1.5 mW/cm² to 200 mW/cm²,
and
a third period of time of 30 to 90 seconds a third plurality of wavelength ranges and radiant powers of:
   a sixth wavelength range is from 400 to 430 nm and a sixth radiant power is between 2 mW/cm² to 200 mW/cm²,
   the fourth disinfecting wavelength range is from 610 nm to 660 nm and the fourth radiant power is between 0.5 mW/cm² to 200 mW/cm², and
   the fifth wavelength range is from 820 nm to 900 nm and the fifth radiant power is between 1.5 mW/cm² to 200 mW/cm²,
wherein respective radiant power is a radiant power at the wound.

The aforementioned second aspect and the third aspect provide a treatment apparatus for treating the chronic wound of the first type successfully. Herein, particular wavelength ranges offer to soothe skin reactions. The processor of the treatment apparatus is configured to control the light source to emit light in such a manner that the particular wavelength ranges that are necessary for healing the chronic wound of the first type, which is akin to multiple pharmaceutical doses affecting various stages of healing in a timely and properly dosed manner. The light is emitted in a balanced manner. For instance, the fifth wavelength range of light is emitted with a particular disinfecting wavelength range, because without said disinfecting wavelength range, an infection could be exacerbated, as the fifth wavelength range of light also nourish bacteria. The treatment apparatus is optimized for different types of wounds (and skin diseases), i.e., the chronic wound of the first type and/or the second type, achieving the most effective treatment without a need to manage various treatments and devices. Thus, the treatment apparatus can also be used in conjunction with traditional treatments. Furthermore, using of multiple wavelengths ensures better treatment outcomes with smaller doses.

Throughout the present disclosure, the term “treatment apparatus” refers to an equipment that is configured to treat wounds of patients by photo biomodulation (PBM) therapy. The treatment apparatus is configured to utilise a comprehensive spectrum of wavelengths (as discussed later), ranging from disinfection purposes to accelerated healing and minimal scarring purposes. The treatment apparatus is configured to be used for therapeutic applications. Examples of the therapeutic applications may include, but are not limited to, disinfection, alleviation of pain and itch, stimulation of endorphin production, reduction of swelling, revitalization of blood circulation, activation of immune system, deactivation of immune cells like T-cells and dendritic cells, promotion of function of inflammation-regulating cells, regulation of skin metabolism, stimulation of mitosis, increased cellular energy production (i.e., generation of high amounts of adenosine triphosphate (ATP)), and growth of collagen and epithelial cells by nearly double the amount.

It will be appreciated that the processor is communicably coupled with the light source. Optionally, the processor is communicably coupled with the light detector. The processor could be implemented as any one of: a microprocessor, a microcontroller, or a controller. As an example, the processor could be implemented as an application-specific integrated circuit (ASIC) chip, or a reduced instruction set computer (RISC) chip.

Throughout the present disclosure, the term “light source” refers to an element from which light emanates. The light source is controllable (for example, using electrical signals) to dynamically adjust illumination characteristics of the light emitted therefrom. In other words, the light source supports dynamic illumination and treatment of the wound. Optionally, the light source emits light in at least one of: an infrared spectrum, a visible-light spectrum. Examples of light sources may include, but are not limited to, light-emitting diodes (LEDs), lasers. Herein, the LEDs could be designed to emit light in given wavelength ranges. As an example, light source could be made from multiple LEDs each emitting light in different wavelength ranges such as first led 200-230 nm, a second 230-250 nm, a third led 240-260 nm etc. This way the multiple LEDs can be configured to emit given ranges for example fi need is to emit from 230 to 260 then the second and the third led would be activated at the same time.

The light is administered in at least two phases, namely, a disinfecting phase and a treatment phase, during the first time period and the second time period, respectively. The light is emitted towards the wound, and optionally, a healthy skin portion surrounding the wound. Optionally, the processor is configured to control the light source to emit light lying in a wavelength range from 200 nanometres (nm) to 1000 nm. A radiant power of the light emitted by the light source can be controlled by the processor to administer a required amount of photons towards the wound and optionally the healthy skin portion surrounding the skin, during a given period of time. Herein, the term “given period of time” encompasses the first period of time and/or the second period of time.

Optionally, the treatment apparatus further comprises a light detector, which is arranged to measure reflectance of the light that is emitted. The processor is optionally communicably coupled with the light detector. The light detector is configured to measure reflectance of the light emitted towards the wound, and the healthy skin portion surrounding the wound. Further more the light detector could be used to analyse how the light is absorbed in the wound and surrounding skin area. This can be used to further control light source.

Optionally, the processor of the treatment apparatus is further communicably coupled to a server via a communication network. Herein, the term “server” relates to a structure and/or module that include programmable and/or non-programmable components configured to store, process and/or share information. optionally, the sever includes any arrangement of physical or virtual computational entities capable of enhancing information to perform various computational tasks. Furthermore, it should be appreciated that the server may be both single hardware server and/or plurality of hardware servers operating in a parallel or distributed architecture. In an example, the server may include components such as memory, a processor, a network adapter and the like, to store, process and/or share information with other computing components, such as user device/user equipment. Optionally, the server is implemented as a computer program that provides various services (such as database service) to other devices, modules or apparatus.

Moreover, the term “communication network” relates to an arrangement of interconnected programmable and/or non-programmable components that are configured to facilitate data communication between one or more electronic devices and/or databases, whether available or known at a time of filing or as later developed. Furthermore, the communication network may include, but is not limited to, one or more peer-to-peer network, a hybrid peer-to-peer network, local area networks LANs), radio access networks (RANs), metropolitan area networks (MANS), wide area networks (WANs), all or a portion of a public network such as the global computer network known as the Internet, a private network, a cellular network and any other communication system or systems at one or more locations. Additionally, the network includes wired or wireless communication that can be carried out via any number of known protocols, including, but not limited to, Internet Protocol (IP), Wireless Access Protocol (WAP), Frame Relay, or Asynchronous Transfer Mode (ATM).

Optionally, the processor is further configured to send the measure of the reflectance (and/or absorption) of the light, as measured by the light detector, to the server via the communication network. Herein, this measure of reflectance is used for determining a treatment parameter from amongst a plurality of treatment parameters, wherein the treatment parameter is to be used for the treatment of the wound. Hence, the treatment apparatus can provide over-the-air (OTA) services, thereby updating the disinfecting phase and the treatment phase as they evolve.

Moreover, the processor is further configured to perform analysis, enable measurement of efficacy and suitability of the treatment of the wound, and provide future guidance based on artificial intelligence.

Throughout the present disclosure, the term “period of time” refers to a duration (namely, a length) of time between a start time and an end time, during which the wound is exposed to the light emitted from the light source. Thus, the first period of time is a duration of time between time instants t11 and t12, whereas the second period of time is a duration of time between time instants t21 and t22. The given period of time may be expressed in terms of millisecond (ms), second(s), minute (min), or similar. In proffered embodiment the second period of time is right after the first period of time i.e. there is no time difference or only small-time difference (0.1 sec, 1 sec, 5 sec, 10 sec, 30 sec) between the first period of time and the second period of time. Preferably time difference between the first period and the second period (and third period) does not exceed 30 sec in order to keep wound area non disinfected.

The processor is configured to control the light that is to be administered in the disinfecting phase. Herein, the light source emits the first plurality of wavelength ranges, i.e., a spectrum of wavelength ranges, wherein the first plurality of wavelength ranges is from wavelength I11 to wavelength I12. The first plurality of wavelength ranges is emitted by the light source in either a continuous sequence or pulsed at a plurality of frequencies. Alternatively, the first plurality of wavelength ranges is emitted by the light source as multiple wavelengths simultaneously, either in a continuous sequence or pulsed at a plurality of frequencies.

Optionally, the plurality of frequencies lies in a range of 10 hertz (Hz) to 100 Hz, 500 to 1000 Hz, or 2000 to 5000 Hz. When pulsing the light source a duty ratio of 50:50 is typically employed.

The first plurality of wavelength ranges comprises the first disinfecting wavelength range, which is useful for disinfection purposes. A technical benefit of emitting the first plurality of wavelength ranges is to at least partially eliminate harmful microorganisms (for example, such as bacteria, virus, fungi, and the like) from the wound, or from the healthy skin portion surrounding the wound. For example, certain wavelengths of light can be antimicrobial and may be used to kill the microorganisms or other pathogens. This at least partial elimination of harmful microorganisms aids in enhancing an efficacy of light emitted from the light source, especially when a response of the immune system is regulated at an onset of the treatment phase.

During the given period of time, a total power of light emitted from the light source is termed as a “given radiant power”. Herein, the given radiant power encompasses the first radiant power and/or the second radiant power. Herein, units of measurement of the given radiant power is microwatts (uW) per square centimetre (cm²), milliwatts (mW) per square centimetre (cm²), etc. at a site of the wound. In this regard, the light emitted from the light source forms a dose in terms of energy per unit area (a unit of measurement is Joules (J)/cm²) which is derived from a power output of the light source per unit area and the given period of time. Depending on a type of wound, the given radiant power is adjusted to disinfect or treat the wound based on a distance of the wound from the light source. It will be appreciated that when the given radiant power is low, it can be compensated by increasing a time required for disinfecting the wound or treating the wound.

Herein, the first radiant power indicates an intensity of the light reaching the wound for disinfecting the wound, during the first period of time. In this regard, when the treatment apparatus is used for treating the wound, a distance between the light source and the wound is measured. The first radiant power of the light source is adjusted based on this distance, to achieve the first radiant power that is required at the site of the wound (for example, such as at surface of skin). This adjustment ensures that the light comprising the first plurality of wavelength ranges effectively reaches the wound with an appropriate intensity to disinfect the wound. A technical benefit of meticulously controlling the light emitted from the light source in such a manner is that it enables disinfection at the site of the wound, enhancing efficacy and efficiency in treatment of the wound.

The processor is configured to control the light to be administered in the treatment phase. Herein, the light source emits the second plurality of wavelength ranges, wherein the second plurality of wavelength ranges is from wavelength I21 to wavelength I22. The second plurality of wavelength ranges comprises the second disinfecting wavelength range for treating the wound, or the first disinfecting wavelength range for disinfecting purposes while treating the wound. A technical benefit of configuring the processor to control the light source in such a manner is that the wound can be treated, or remaining harmful microorganisms, which was not eliminated during the first period of time, can be eliminated during the second period of time.

Herein, the second radiant power indicates an intensity of the light reaching the wound for either treating the wound, or disinfecting the wound, during the second period of time. In this regard, the second radiant power of light is adjusted in a same manner as is described above for the first radiant power of light. A technical benefit of meticulously controlling the light emitted from the light source in such a manner is that it enables precise and tailored treatment strategies, enhancing efficacy and efficiency in treatment of the wound.

Optionally, the first disinfecting wavelength range is from 200 nm to 280 nm and the first radiant power is between 50 uW/cm² to 1 mW/cm²;
the second disinfecting wavelength range is from 300 nm to 390 nm and the second radiant power is between 100 uW/cm² to 25 mW/cm²,
wherein each respective radiant power is a radiant power of respective wavelength range of light at the wound.

Herein, the first disinfecting wavelength range and the second disinfecting wavelength range lies in an ultraviolet (UV) light spectrum. It will be appreciated that the first disinfecting wavelength range and the second disinfecting wavelength range primarily affects outer layers of the skin, which is beneficial for treating wounds without causing damage to tissues. Hence, light emitted in the first disinfecting wavelength range and the second disinfecting wavelength range is effective for treating skin conditions that are confined to surface layers of the skin.

The first disinfecting wavelength range lies in UV-C region of the UV light spectrum, and the second disinfecting wavelength range lies in UV-A region of the UV light spectrum. The first disinfecting wavelength range may be, for example, from 200, 210, 220, 240, or 260nm to 230, 250, 270, or 280 nm. The first radiant power may lie in a range, for example, from 50×10⁻⁶, 60×10⁻⁶, 80×10⁻⁶, or 100×10⁻⁶ W/cm² to 90×10⁻⁶, 500×10⁻⁶, 500×10⁻⁶, 800×10⁻⁶, 900×10⁻⁶, 1 mW/cm². A technical benefit of the first disinfecting wavelength range is that it has potent disinfecting properties, anti-inflammatory properties, and wound-healing properties. Hence, the first disinfecting wavelength range is used to at least one of: reduce inflammation of wounds by promoting the function of the inflammation-regulating cells and release of inflammatory mediators, promote healing of the wound by stimulation of collagen (an essential protein that forms structure of the skin and aids in wound healing) production, at least partially eliminate the microorganisms, sterilize wounds, reduce a risk of infection. It has been found out that if (any of the) radiant power(s) is too low then there is not sufficient effect and/or treatment time is too long. Also if the radiant power is too high then light might cause uncomforting feeling or damage the skin.

The second disinfecting wavelength range may be, for example, from 300, 310, 320, 340, or 360 nm to 330, 350, 370, 380, or 390 nm. The second radiant power may lie in a range, for example, from 100 uW/cm², 200 uW/cm², 400 uW/cm², 1 mW/cm², 10 mW/cm², or 20 mW/cm² to 300 uW/cm², 500 uW/cm², 5 mW/cm², 15 mW/cm², or 25 mW/cm². A technical effect of the second disinfecting wavelength range is that it can be used for deactivate the immune system in the skin, selective phototherapy, reduce inflammation, soothe skin symptoms. Hence, the second disinfecting wavelength range is used for various skin diseases such as for example, psoriasis, eczema, and vitiligo. (Terms: uW=microwatt=10⁻⁶ W, mW=milliWatt=10⁻³ W)

Optionally, the processor is configured to control the light source to emit another disinfecting wavelength range, wherein the another disinfecting wavelength range is between 280 nm to 320 nm. Herein, the another disinfecting wavelength range also lies in an ultraviolet (UV) light spectrum. The another disinfecting wavelength range lies in UV-B region of the UV light spectrum. The another disinfecting wavelength range may be, for example, from 280, 290, or 310 nm to 285, 305, 315, or 320 nm. Herein, the another disinfecting wavelength range has anti-inflammatory properties and can be used to treat inflammatory skin diseases (for example, such as different types of psoriasis, eczema, etc.). The light emitted of the another disinfecting wavelength range affects the immune system and reduces autoimmune reactions. Furthermore, said light of the another disinfecting wavelength range can influence immune cells, such as T-cells, which play a central role in the development of many skin diseases.

In an embodiment, the first plurality of wavelength ranges comprises the first disinfecting wavelength range of light having the first radiant power, the second disinfecting wavelength range of light having the second radiant power, and a third disinfecting wavelength range of light having a third radiant power; and

the second plurality of wavelength ranges comprises the first disinfecting range of wavelengths of light having the first radiant power, a fourth disinfecting range of wavelengths of light of a fourth radiant power and a fifth range of wavelengths of light from of a fifth radiant power.

Herein, the first plurality of wavelength ranges are used for disinfecting the site of the wound. The first disinfecting wavelength range of light and the second disinfecting wavelength range of light have already been discussed above. The third disinfecting wavelength range of light provides a comprehensive approach for disinfection purposes, wherein the first plurality of wavelength ranges potentially targets a broad spectrum of microorganisms or optimizes an efficacy of disinfecting the wound.

Herein, the third radiant power indicates an intensity of the light reaching the wound for disinfecting the wound, during the first period of time. In this regard, the third radiant power is adjusted in a same manner as is described above for the first radiant power of light.

The second plurality of wavelength ranges are used for treating the wound or disinfecting the wound. Herein, the first disinfecting wavelength range is used for disinfecting the wound. Moreover, the fourth disinfecting range of wavelengths of light and the fifth range of wavelengths of light are used for treating the wound. Herein, the fourth radiant power and the fifth radiant power indicates respective intensities of the light reaching the wound for treating the wound, during the second period of time. In this regard, the fourth radiant power and the fifth radiant power are adjusted in a same manner as is described above for the first radiant power of light.

A technical effect of combining the wavelengths in the first plurality of wavelengths and the second plurality of wavelengths is that a comprehensive treatment is achieved that addresses multiple skin layers, from an outer layer (namely, epidermis) of the skin to a deepest layer (namely, a hypodermis) of the skin. Such combination beneficially improves a health of the skin across the outer layer to the deepest layer, and also provides effective disinfection from the surface of the skin down to a depth of 4 millimetres (mm).

Optionally, the third disinfecting wavelength range is from 400 nm to 430 nm and the third radiant power is between 3 mW/cm² to 200 mW/cm², wherein respective radiant power is a radiant power at the wound. Herein, the third disinfecting wavelength range lies within blue region of visible light spectrum. It will be appreciated that the third disinfecting wavelength range is beneficial in treatment of acne as it kills Propionibacterium acnes bacteria, which is associated with the development of acne. The third disinfecting wavelength range excites endogenous light-sensitive chromophores in microbial cells, leading to generation of reactive oxygen species (ROS) that are toxic to microbial cells. The third disinfecting wavelength range may, for example, lie from 400, 405, 410, or 420 nm to 402, 415, 425, or 430 nm. The third radiant power may, for example, lie from 3, 10, 20, 50, 100, or 180 mW/cm² to 70, 110, 140, 170, 190, or 200 mW/cm². A technical benefit of the third disinfecting wavelength range is that it can be used to treat microbial infections that has few or no side effects on humans or animals. The respective radiant power refers to the intensity of light measured specifically at the site of the wound. Hence, it is crucial to accurately deliver an appropriate amount of light energy to the wound to achieve a therapeutic effect that is required.

Optionally, the fourth disinfecting wavelength range is from 610 nm to 660 nm and the fourth radiant power is between 0.5 mW/cm² to 200 mW/cm²; and

the fifth wavelength range is from 820 nm to 900 nm and the fifth radiant power is between 1.5 mW/cm² to 200 mW/cm², wherein each respective radiant power is a radiant power of respective wavelength range of light at the wound.

Herein, the fourth disinfecting wavelength range lies within red region of the visible light spectrum, and the fifth wavelength range lies in a near-infrared region of electromagnetic light spectrum, which lies beyond the visible light spectrum. The fourth disinfecting wavelength range and/or the fifth wavelength range can be used for treatment of at least one of: the outer layer of the skin, dermis (wherein the dermis is located beneath the epidermis and above a subcutaneous layer), sebaceous gland, the hypodermis.

When the disinfecting fourth wavelength range of the fourth radiant power and/or the fifth wavelength range of the fifth radiant power is used for treatment of the outer layer of the skin, production of collagen and epithelial cells are stimulated which boosts cell metabolism in lower layers of the epidermis. This facilitates at least one of: skin rejuvenation, skin firming, and diminish fine lines and wrinkles. Furthermore, the fourth disinfecting wavelength range or the fifth wavelength range offers protection to the wounds, enhance healing of wounds, and reduce inflammation. Thereby, this aids in decreasing a quantity of dead cells or damaged cells, and encourages development of new cells at the site of the wound. A technical benefit is that it contributes to reduction of a size of the wound, thereby at least one of: promoting closure, minimizing scarring, of the wound. Beneficially, this also provide relief from any potential itching or discomfort.

When the fourth disinfecting wavelength range of the fourth radiant power and/or the fifth wavelength range of the fifth radiant power is used for treatment of the dermis, the light is penetrated deeper into the dermis to enhance blood circulation. This promotes delivery of oxygen and nutrients to the cells and aids in removal of waste products. Additionally, fibroblasts can be stimulated which leads to production of collagen, wherein the production of collagen helps strengthen the skin's structure and improve an appearance of scars. Furthermore, the fifth range of wavelengths aid in alleviating wound pain by affecting nerve function and potentially reducing the transmission of pain signals.

When the fourth disinfecting wavelength range of the fourth radiant power and/or the fifth wavelength range of the fifth radiant power is used for treatment of the sebaceous gland, an oiliness of the skin can be decreased by reducing an activity of the sebaceous gland, thus preventing development of acne.

When the fourth disinfecting wavelength range of the fourth radiant power and/or the fifth wavelength range of the fifth radiant power is used for treatment of the hypodermis, metabolism of the cells is impacted upon which stimulates tissue healing. Herein, the fourth radiant power and/or the fifth radiant power is absorbed by the cells, which affects the mitochondria. The fourth disinfecting wavelength range and/or the fifth wavelength range can stimulate collagen production by up to 170% compared to normal collagen production, in subcutaneous fat tissue. It will be appreciated that the collagen is a crucial protein that forms connective tissue and aids in wound closure. Furthermore, the fourth disinfecting wavelength range and/or the fifth wavelength range can help alleviate pain around the wound by influencing nerve function and reducing the transmission of pain signals. Moreover, the fourth disinfecting wavelength range and/or the fifth wavelength range dilates blood vessels and improves circulation in the wounded area, by increasing nutrient and oxygen supply to the tissue, and thus promoting healing. Such circulation in the wounded area can reduce inflammation in tissues of the wound, which affects the immune system and reduces an intensity of the inflammatory response, thus helping to prevent excessive swelling and redness.

The fourth disinfecting wavelength range may, for example, lie in a range from 610, 615, 620, 630, or 650 nm to 625, 645, 655, or 660 nm. The fourth radiant power may, for example, lie between 0.5, 1, 10, 20, 50, 100, or 150 mW/cm² to 10, 20, 30, 70, 90, 110, 115, or 200 mW/cm². The fifth wavelength range may, for example, lie in a range from 820, 825, 830, 840, or 870 nm to 850, 880, 890, 895, or 900 nm. The fifth radiant power may, for example, lie in a range from 1.5, 2, 10, 20, 40, 80, 150 mW/cm² to 10, 20, 40, 50, 110, 160, 180, 190, or 200 mW/cm². A technical benefit of emitting light of different wavelengths is that such light has distinct penetration abilities into various tissue layers, offering a range of benefits and therapeutic effects. In general, for disinfecting light wave ranges it is important to keep a minimum level of radiative power on during the treatment phases. On the otherhand, especially, during second (or third) period of time level should not exceed provided radiant range as it might have negative impact on healing process.

Optionally, the fourth radiant power is provided as pulses having a frequency of 80 Hz to 120 Hz and with a duty ratio of ON:OFF from 30:70 to 70:30. This frequency of the fourth radiant power represents a number of pulses emitted per second. The frequency may, for example, lie in a range of 80, 85, 90, or 110 Hz to 95, 105, 115, or 120 Hz. The term “duty ratio” refers to a proportion of time during which the fourth radiant power is actively ON compared to a time when the fourth radiant power is OFF (namely, inactive). Herein, the duty ratio is expressed as a ratio which represents a percentage of time the fourth radiant power is ON versus OFF during each pulse cycle. As an example, a duty ratio of 30:70 may mean that the fourth radiant power is ON for 30 percent of the pulse cycle, and OFF for remaining 70 percent of the pulse cycle. As another example, a duty ratio of 70:30 may mean that the fourth radiant power is ON for 70 percent of the pulse cycle, and OFF for remaining 30 percent of the pulse cycle. A technical effect of providing the fourth radiant power in such a manner is that it facilitates precise control over a timing and intensity of the light emitted from the light source, thereby potentially optimizing therapeutic effects for various applications (as listed above). Preferably the duty ratio is 50:50 but the duty ratio can be optionally for example 20:80, 30:70, 40:60, 60:40, 70:30 or 80:20 in which first number is ON time and second is OFF i.e. for example 30:70 means 30% ON and 70% OFF. Technical benefit of this is to allow cells to recover for small moments of time (during OFF time). This has been found out to improve wound healing in some cases.

In another embodiment, the first plurality wavelength ranges comprises the first disinfecting wavelength range of light having the first radiant power, a fourth disinfecting wavelength range of light having a fourth radiant power, and the fifth wavelength range of light having the fifth radiant power; and

the second plurality of wavelength ranges comprises the second disinfecting wavelength range of light having the second radiant power, the fourth disinfecting range of wavelength of light of the fourth radiant power and the fifth range of wavelength of light from of the fifth radiant power.

Herein, in both the first period of time and the second period of time, the processor is configured to control the light source in such a manner that the wound is disinfected and/or treated. Furthermore, this is beneficial for generation of the ATP, and for pain relief. The first plurality of wavelengths and the second plurality of wavelengths targets a broad spectrum of microorganisms or optimizes an efficacy of disinfecting and treating the wound during the first period of time and the second period of time.

Optionally, the processor is further configured to control the light source to emit:

during a third period of time, a light comprising a third plurality of wavelength ranges, wherein the third plurality of wavelength ranges comprises a sixth range of wavelength of lights having a sixth radiant power, the fourth disinfecting range of wavelength of lights having the fourth radiant power and the fifth range of wavelength of lights having the fifth radiant power. Herein, the processor is further configured to control the light to be administered post the treatment phase, wherein the third plurality of wavelength ranges is from wavelength I31 to wavelength I32. The third plurality of wavelength ranges is emitted by the light source in either a continuous sequence or pulsed at the plurality of frequencies (as described above). Alternatively, the first plurality of wavelength ranges is emitted by the light source as multiple wavelengths simultaneously, either in a continuous sequence or pulsed at the plurality of frequencies. Herein, the third period of time is a duration of time between time instants t31 and t32. Moreover, combining the sixth range of wavelength of lights having a sixth radiant power, the fourth disinfecting range of wavelength of lights having the fourth radiant power and the fifth range of wavelength of lights having the fifth radiant power is that such combination can soothe any reactions on the skin. Additionally, such combination is used for at least one of: producing corrective effects, increasing production of epithelial tissue, increasing production of collagen, accelerate mitosis, release endorphins, enhance oxygen-carrying capacity of haemoglobin, enhance blood circulation, enhance metabolism.

Optionally, the sixth wavelength range is from 400 to 430 nm and the sixth radiant power is between 2 mW/cm² to 200 mW/cm², wherein respective radiant power is a radiant power at the wound. Herein, the sixth wavelength range lies within the blue region of the visible light spectrum. A technical effect of the sixth wavelength range is that it is utilised for treating the skin post the treatment phase, wherein the sixth wavelength range is used for post treatment of at least one of: the dermis, the sebaceous glands. When the sixth wavelength range of the sixth radiant power is used for the post treatment of the dermis, it reduces pain, inflammation, and redness in the dermis. When the sixth wavelength range of the sixth radiant power is used for the treatment of the sebaceous glands, symptoms of acne is reduced by influencing the sebaceous glands to diminish their activity, and thereby reducing sebum production. The sixth wavelength range may, for example, lie from 400, 405, 410, or 420 nm to 402, 415, 425, or 430 nm. The sixth radiant power may, for example, lie from 2, 10, 20, 50, 100, or 180 mW/cm² to 70, 110, 140, 170, 190, or 200 mW/cm².

Optionally, the first time period is between 30 to 90 seconds, the second time period is between 30 to 90 seconds and the third time period is between 30 to 90 seconds. A technical effect of the first time period, the second time period, and the third time period to lie between 30 to 90 seconds is that a minimum requirement of the given radiant power can be reached to have the therapeutic effect at the wound. The first time period, the second time period, and the third time period may, for example, lie in a range from 30, 35, 40, 50, or 70 seconds to 45, 60, 80, 85, or 90 seconds.

Optionally, different ranges of wavelengths do not overlap with each other and have a wavelength range gap between each neighbouring ranges. In this regard, when considering the light emitted by the light source, there are clear boundaries separating the different ranges of wavelengths from each other, and there are no shared wavelengths between adjacent wavelength ranges. Herein, the term “wavelength range gap” refers to a noticeable difference in wavelength between each neighbouring range. Such wavelength range gaps ensures that each range of wavelengths that are emitted by the light source serves a particular purpose without interference or overlap from the neighbouring range. A technical effect of the different ranges of wavelengths not overlapping with each other facilitates targeting of desired ranges of wavelengths for particular applications, i.e., for therapeutic purposes and/or disinfecting purposes, without a risk of unwanted or unintended effects due to any overlapping. A gap can be for example from 5 nm, 10 nm, 15 nm, 20 nm, 25 nm, 30 nm, 40 nm, 50 nm, 60 nm, 70 nm, 80 nm, 90 nm, 100 nm up to 10 nm, 15 nm, 20 nm, 25 nm, 30 nm, 40 nm, 50 nm, 60 nm, 70 nm, 80 nm, 90 nm, 110 nm. Range of wavelengths, when emitted can refer to a spectrum in which intensity value from maximum peak value of the spectrum are ½ (half peak width value) I.e if peak has maximum value at 500 nm and ½ of maximum at 450 nm and 520 nm then the wavelength of said light emitter (LED or set of LEDs or light source as it is configured at said moment) can be deemed to emit light from 450 to 520 nm. Alternative other values than ½ value can be used such as ⅓, ¼, ⅕, ⅙, ⅛, 1/9 as an example.

Optionally, the treatment apparatus is configured to control the light source to emit light according to claims 1, 2, 5, 6, and 7 when a wound type is a diabetes related wound. Herein, the treatment apparatus is configured to emit light of particular disinfecting wavelength ranges of light and particular ranges of wavelengths of light for the disinfecting phase and treatment phase of the wound, when the wound type is a diabetes related wound. Herein, the term “diabetes related wound” refers to the wound type that occurs as a complication of diabetes mellitus. Herein, the diabetes related wound results from at least one of: impaired blood circulation, nerve damage (neuropathy), compromised immune function, which are commonly associated with diabetes. Examples of such diabetes related wounds may include, but are not limited to, diabetic foot ulcers, neuropathic wounds, and charcot foot.

Optionally, the treatment apparatus is configured to control the light source to emit light according to claims 1, 3, 4, 7, and 8 when the wound type is a diabetes related wound. Herein, the treatment apparatus is configured to emit light of particular disinfecting wavelength ranges of light and particular ranges of wavelengths of light for the disinfecting phase and treatment phase of the wound, when the wound type is a diabetes related wound.

The present disclosure also relates to a computer software, wherein the computer software is configured to run in a processor to cause the processor to control a light source, according to the aforementioned first aspect. Various embodiments and variants disclosed above, with respect to the aforementioned first aspect, apply mutatis mutandis to the computer software.

Herein, the term “computer software” refers to a software program executed on a use device by employing the processor of said user device. Notably, the computer software comprises a set of predefined functions therein that are programmed to provide instructions to hardware and/or software elements of the user device. Furthermore, the computer software is configured to provide a user interface on the display of the user device, to allow the user to perform specific tasks associated with the aforementioned method. In an example, the computer software is an application programming interface. In an embodiment, the computer software is affiliated to an organisation. Therefore, the computer software functions in accordance with pre-programmed guidelines provided by the organisation. The computer software is configured to function in accordance with the pre-programmed guidelines upon installation thereof.

The present disclosure also relates to the second aspect as described above. Various embodiments and variants disclosed above, with respect to the aforementioned first aspect, apply mutatis mutandis to the second aspect.

The present disclosure also relates to the third aspect as described above. Various embodiments and variants disclosed above, with respect to the aforementioned first aspect, apply mutatis mutandis to the third aspect.

### EXPERIMENTAL PART

### Experimental Example 1

The treatment apparatus was used for treatment of a diabetes related wound. Herein, the patient was a woman of at least 70 years of age with weakened blood circulation in her legs due to diabetes was facing a threat of above-knee amputation of both legs due to severe pain and the ineffectiveness of medications. The processor of the treatment apparatus was configured to control the light source in such a manner that a Recipe 1 was devised for treating the diabetes related wound. After a few treatments according to the Recipe 1, her pain disappeared, and after approximately ten treatments, her blood circulation improved to the extent that amputation was no longer considered necessary. Additionally, one of her toes, which had been classified as lost, was preserved. However, when treating with different parameters outside of the Recipe 1, no positive results were available for the diabetes related wound.

In the Recipe 1, the processor was configured to control the light source to emit:
during a first period of time (namely, the disinfecting phase), a light comprising a first disinfecting wavelength range having a first radiant power, a second disinfecting wavelength range having a second radiant power, and a third disinfecting wavelength range having a third radiant power;
during a second period of time (namely, the treatment phase), a light comprising the first disinfecting wavelength range having the first radiant power, a fourth disinfecting wavelength range having a fourth radiant power, and a fifth wavelength range having a fifth radiant power; and
during a third period of time (namely, post-treatment phase), a light comprising the first disinfecting wavelength range having the first radiant power, the fourth disinfecting wavelength range having the fourth radiant power, and the fifth wavelength range having the fifth radiant power.

The disinfecting phase was carried out for a skin depth lying in a range of 1 millimetre (mm) to 4 mm, the treatment phase was carried out for a skin depth of 1 mm, and the post-treatment phase was carried out for a skin depth of 1 mm. The treatment phase provided pain relief to the woman, and generated ATP. The post-treatment phase produced corrective effects, increasing production of epithelial tissue by 140 percent of normal production of epithelial tissues, increasing production of collagen by 180% of the normal production of collagen, accelerating mitosis, releasing endorphins, enhancing oxygen-carrying capacity of haemoglobin, enhancing blood circulation, enhancing metabolism. Moreover, the first period of time, the second period of time, and the third period of time was for 60 seconds. The Recipe 1 is provided in Table 1,

TABLE 1

| | First period of | Second period of | Third period of |
|---|---|---|---|
| Time | time | time | time |
| Note 1 | Disinfecting 1- | Disinfecting | Disinfecting 1 mm |
| | 4 mm skin depth | 1 mm skin depth | skin depth |
| Note 2 | | Cellular ATP, | Corrective, |
| | | pain relief | epithelial + |
| | | | 140%, collagen + |
| | | | 180%, + |
| | | | mitosis, |
| | | | endorphin, |
| | | | oxygenation of |
| | | | haemoglobin, |
| | | | enhancement of |
| | | | blood circulation |
| | | | and metabolism. |
| Wavelength | First disinfecting | First disinfecting | First disinfecting |
| range | wavelength | wavelength | wavelength |
| | range (200- | range (200- | range (200- |
| | 280 nm) | 280 nm) | 280 nm) |
| Radiant | First radiant | First radiant | First radiant |
| power | power (50 uW- | power (50 uW- | power (50 uW- |
| | 1 mW) | 1 mW) | 1 mW) |
| Wavelength | Second | Fourth | Fourth |
| range | disinfecting | disinfecting | wavelength |
| | wavelength | wavelength | range (610- |
| | range (300- | range (610- | 660 nm) |
| | 390 nm) | 660 nm) | |
| Radiant | Second radiant | Fourth radiant | Fourth radiant |
| power | power (100 uW- | power (0.5 mW- | power (0.5 mW- |
| | 25 mW) | 200 mW) | 200 mW) |
| Wavelength | Third | Fifth wavelength | Fifth wavelength |
| range | disinfecting | range (820- | range (820- |
| | wavelength | 900 nm) | 900 nm) |
| | range (400- | | |
| | 430 nm) | | |
| Radiant | Third radiant | Fifth radiant | Fifth radiant |
| power | power (3 mW- | power (1.5 mW- | power (2 mW- |
| | 200 mW) | 200 mW) | 200 mW) |

### Experimental Example 2

The treatment apparatus was used for treatment of chronic diabetes related wound. Herein, the patient was a 64-year-old man with diabetes having several chronic wounds on the soles of his feet. He had only two toes remaining, and the wounds threatened further losses in his feet. The oldest wound was over a year old and had not healed, similarly to other wounds that were more than three months old. The processor of the treatment apparatus was configured to control the light source in such a manner that a Recipe 2 was devised for treating the chronic diabetes related wound. After a few treatments according to the Recipe 1, his oldest wound closed and healed, as did more recent wounds.

In the Recipe 2, the processor was configured to control the light source to emit:
during a first period of time, a light comprising a first disinfecting wavelength range having a first radiant power, a fourth disinfecting wavelength range having a fourth radiant power, a fifth range of wavelength of lights having a fifth radiant power;
during a second period of time, a light comprising a second disinfecting wavelength range having a second radiant power, the fourth disinfecting wavelength range having the fourth radiant power, the fifth range of wavelength of lights having the fifth radiant power; and
during a third period of time, a light comprising a sixth range of wavelength of lights having a sixth radiant power, the fourth disinfecting wavelength range having the fourth radiant power, the fifth range of wavelength of lights having the fifth radiant power.

The disinfecting phase was carried out for a skin depth for a skin depth of 1 mm, the treatment phase was carried out for a skin depth of 2 mm, and the post-treatment phase was carried out autoinflammation at a skin depth of 4 mm. The disinfecting phase and the treatment phase was carried out for providing pain relief to the man, and for generating ATP, and the post-treatment phase was carried out for accelerating mitosis, releasing endorphins, enhancing oxygen-carrying capacity of haemoglobin, enhancing blood circulation, enhancing metabolism. Moreover, the first period of time, the second period of time, and the third period of time was for 60 seconds. The Recipe 2 is provided in Table 2,

TABLE 2

| | | | |
|---|---|---|---|
| Time | First period of | Second period of | Third period of |
| | time | time | time |
| Note 1 | Disinfecting | Disinfecting | Autoinflammatory |
| | 1 mm skin depth | 2 mm skin depth | 4 mm skin depth |
| Note 2 | Cellular ATP, | Cellular ATP | Mitosis, |
| | pain relief | pain relief | endorphin, |
| | | | oxidation of |
| | | | haemoglobin, |
| | | | enhancement of |
| | | | blood circulation |
| | | | and metabolism. |
| Wavelength | First disinfecting | Second | Sixth range of |
| range | wavelength | disinfecting | wavelength (400- |
| | range (200- | wavelength | 430) |
| | 280 nm) | range (300- | |
| | | 390 nm) | |
| Radiant | 50 uW-1 mW | 100 uW-25 mW | 2 mW-200 mW |
| power | | | |
| Wavelength | Fourth | Fourth | Fourth |
| range | disinfecting | disinfecting | disinfecting |
| | wavelength | wavelength | wavelength range |
| | range | range | (610-660 nm) |
| | (610-660 nm) | (610-660 nm) | pulsed with 100 Hz |
| | pulsed with | pulsed with | with 50:50 duty |
| | 100 Hz with | 100 Hz with | ratio |
| | 50:50 duty ratio | 50:50 duty ratio | |
| Radiant | 10 mW-200 mW | 10 mW-200 mW | 10 mW-200 mW |
| power | | | |
| Wavelength | Fifth range of | Fifth range of | Fifth range of |
| range | wavelength | wavelength | wavelength (820- |
| | (820-900 nm) | (820-900 nm) | 900 nm) pulsed |
| | pulsed with | pulsed with | with 100 Hz with |
| | 100 Hz with | 100 Hz with | 50:50 duty ratio |
| | 50:50 duty ratio | 50:50 duty ratio | |
| Radiant | 10 mW-200 mW | 10 mW-200 mW | 10 mW-200 mW |
| power | | | |

### DETAILED DESCRIPTION OF THE DRAWINGS

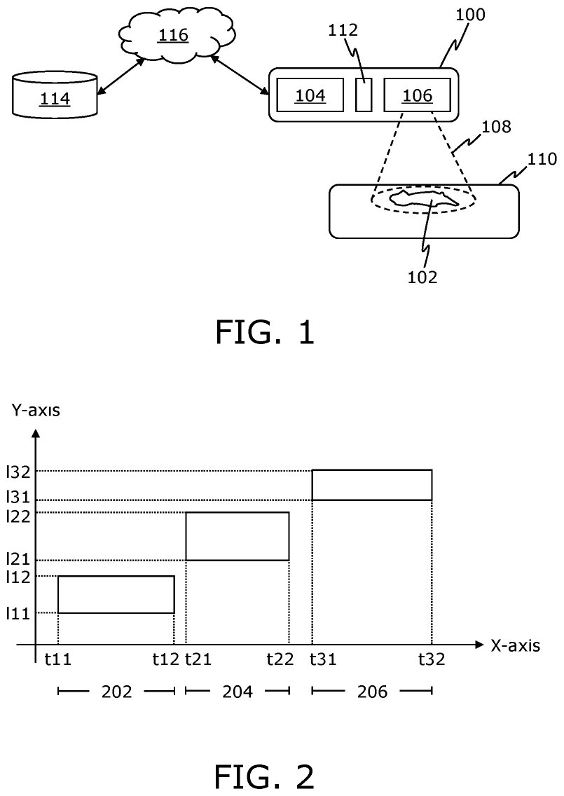

Referring to FIG. 1, illustrated is a schematic illustration of a treatment apparatus 100 for treatment of a wound 102, in accordance with an embodiment of the present disclosure. The treatment apparatus 100 comprises a processor 104 and a light source 106. The light source 106 is arranged to emit light 108 towards the wound 102 when in use. The processor 104 is configured to control the light source 106 to emit during different periods of time, lights comprising different plurality of wavelength ranges, for the treatment of the wound 102. The wound 102 is on and/or under a skin 110 as shown. The treatment apparatus 100 optionally also comprises a light detector 112 arranged to measure reflectance of the light 108 that is emitted. The treatment apparatus 100 (and particularly, the processor 104) is optionally communicably coupled to a server 114 via a communication network 116.

FIG. 1 is merely an example, which should not unduly limit the scope of the claims herein. A person skilled in the art will recognize many variations, alternatives, and modifications of embodiments of the present disclosure. Referring to FIG. 2, illustrated is an exemplary timeline of treatment of a wound, in accordance with an embodiment of the present disclosure. In the exemplary timeline, X-axis represents time and Y-axis represents wavelength.

During a first period of time, depicted as a first period 202 of time between time instants t11 and t12, a light comprising a first plurality of wavelength ranges is emitted towards the wound, wherein at least one of the first plurality of wavelength ranges is a first disinfecting wavelength range of light having a first radiant power. The first plurality of wavelength ranges is from wavelength I11 to wavelength I12.

During a second period of time, depicted as a second period 204 of time between time instants t21 and t22, a light comprising a second plurality of wavelength ranges is emitted towards the wound, wherein at least one of the second plurality of wavelength ranges is the first disinfecting wavelength range of lights having the first radiant power or a second disinfecting wavelength range of light having a second radiant power. The second plurality of wavelength ranges is from wavelength I21 to wavelength I22.

During a third period of time, depicted as a third period 206 of time between time instants t31 and t32, a light comprising a third plurality of wavelength ranges is emitted towards the wound, wherein the third plurality of wavelength ranges comprises a sixth range of wavelength of lights having a sixth radiant power, a fourth disinfecting range of wavelength of lights having a fourth radiant power and a fifth range of wavelength of lights having a fifth radiant power. The third plurality of wavelength ranges is from wavelength I31 to wavelength I32.

The first period of time is before the second period time. The second period time is before the third period of time. Between the period of times there is no or only few second pause i.e. the treatment is essentially continuous.

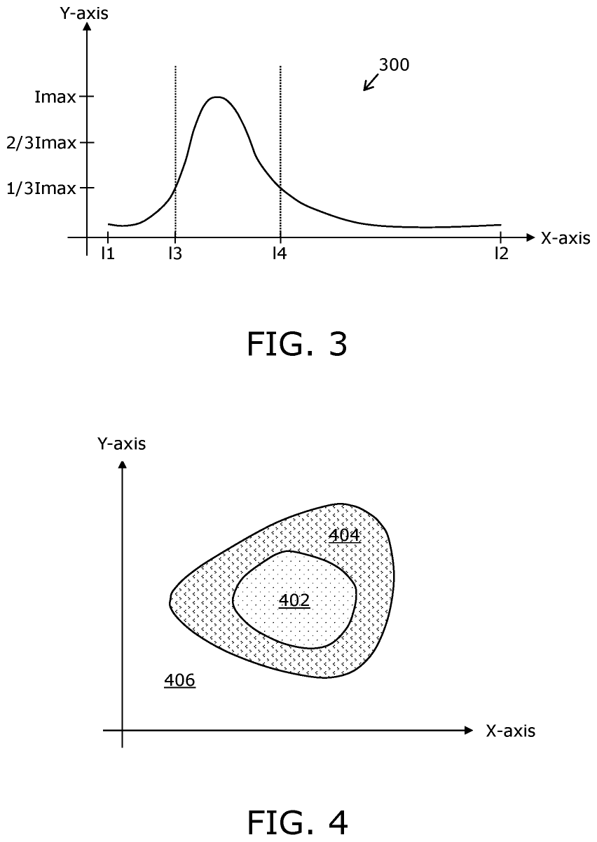

FIG. 2 is merely an example, which should not unduly limit the scope of the claims herein. A person skilled in the art will recognize many variations, alternatives, and modifications of embodiments of the present disclosure. Referring to FIG. 3, illustrated is a graphical illustration of an exemplary spectral profile of a given wavelength range 300, in accordance with an embodiment of the present disclosure. In the spectral profile, X-axis represents wavelength and Y-axis represents intensity. Although the given wavelength range 300 encompasses a wide range of wavelengths, depicted as a range of wavelengths between I1 and I2, but the given wavelength range 300 can be defined to comprise those wavelengths which have an intensity that is greater than a predefined intensity threshold. For example, the predefined intensity threshold may be 33.33% of a maximum intensity (Imax) of the wide range of wavelengths between I1 and I2. In this regard, the given wavelength range 300 can be defined to comprise wavelengths between I3 and I4.

FIG. 3 is merely an example, which should not unduly limit the scope of the claims herein. A person skilled in the art will recognize many variations, alternatives, and modifications of embodiments of the present disclosure. Referring to FIG. 4, illustrated is an exemplary contour plot for treatment of a wound, in accordance with an embodiment of the present disclosure. In the exemplary contour plot, X-axis represents wavelength and Y-axis represents a treatment time. The Y-axis could, for example, alternatively represent radiant power or amount of light. A given plurality of wavelength ranges which are best suited for the treatment of the wound, and a range of a given period of time for which light comprising the given plurality of wavelength ranges is to be emitted towards the wound, are shown for example, as a region 402 (depicted as a dotted hatched region) in the exemplary contour plot. Another plurality of wavelength ranges and a corresponding period of time for which the another plurality of wavelength ranges is to be emitted towards the wound, are shown for example, as a region 404 (depicted as a divot hatched region) in the exemplary contour plot. Wavelengths and treatment times which are not yet known to be effective lie in a region 406 (depicted as an un-hatched unbounded region) that is outside of the region 404.

FIG. 4 is merely an example, which should not unduly limit the scope of the claims herein. A person skilled in the art will recognize many variations, alternatives, and modifications of embodiments of the present disclosure. Referring to FIG. 5, illustrated are steps of a method of treatment of a chronic wound of a first type, in accordance with an embodiment of the present disclosure. At step 502, there is provided during a first period of time of 30 to 90 seconds, a first plurality of wavelength ranges and radiant powers of: a first disinfecting wavelength range is from 200 nm to 280 nm and a first radiant power is between 50 uW/cm² to 1 mW/cm², a second disinfecting wavelength range is from 300 nm to 390 nm and a second radiant power is between 100 uW/cm² to 25 mW/cm², and a third disinfecting wavelength range is from 400 nm to 430 nm and a third radiant power is between 3 mW/cm² to 200 mW/cm². At step 504, there is provided during a second period of time of 60 to 180 seconds, a second plurality of wavelength ranges and radiant powers of: the first disinfecting wavelength range is from 200 nm to 280 nm and the first radiant power is between 50 uW/cm² to 1 mW/cm², a fourth disinfecting wavelength range is from 610 nm to 660 nm and a fourth radiant power is between 0.5 mW/cm² to 200 mW/cm², and a fifth wavelength range is from 820 nm to 900 nm and a fifth radiant power is between 1.5 mW/cm² to 200 mW/cm². Each respective radiant power is a radiant power of respective wavelength range of light at the wound.

The aforementioned steps are only illustrative and other alternatives can also be provided where one or more steps are added, one or more steps are removed, or one or more steps are provided in a different sequence without departing from the scope of the claims.

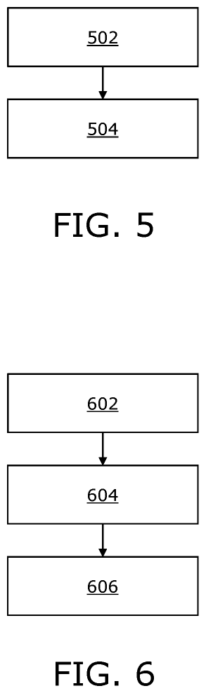

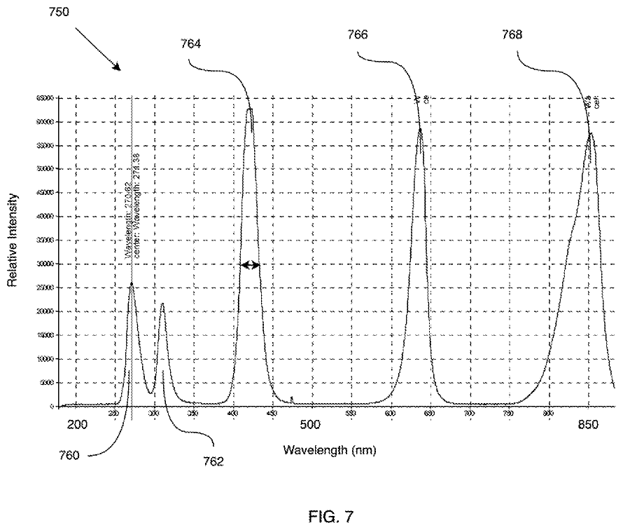

Referring to FIG. 6, illustrated are steps of a method of treatment of a chronic wound of a second type, in accordance with an embodiment of the present disclosure. At step 602, there is provided during a first period of time of 30 to 90 seconds, a first plurality of wavelength ranges and radiant powers of: a first disinfecting wavelength range is from 200 nm to 280 nm and a first radiant power is between 50 uW/cm² to 1 mW/cm², a fourth disinfecting wavelength (fourth wavelength range) range is from 610 nm to 660 nm and a fourth radiant power is between 0.5 mW/cm² to 200 mW/cm², and a fifth wavelength range is from 820 nm to 900 nm and a fifth radiant power is between 1.5 mW/cm² to 200 mW/cm². At step 604, there is provided during a second period of time of 30 to 90 seconds, a second plurality of wavelength ranges and radiant powers of: a second disinfecting wavelength range is from 300 nm to 390 nm and a second radiant power is between 100 uW/cm² to 25 mW/cm², the fourth disinfecting wavelength range is from 610 nm to 660 nm and the fourth radiant power is between 0.5 mW/cm² to 200 mW/cm², and the fifth wavelength range is from 820 nm to 900 nm and the fifth radiant power is between 1.5 mW/cm² to 200 mW/cm². At step 606, there is provided during a third period of time of 30 to 90 seconds a third plurality of wavelength ranges and radiant powers of: a sixth wavelength range is from 400 to 430 nm and a sixth radiant power is between 2 mW/cm² to 200 mW/cm², the fourth disinfecting wavelength range is from 610 nm to 660 nm and the fourth radiant power is between 0.5 mW/cm² to 200 mW/cm², and the fifth wavelength range is from 820 nm to 900 nm and the fifth radiant power is between 1.5 mW/cm² to 200 mW/cm². A respective radiant power is a radiant power at the wound.

FIG. 7. is provides a measured spectra 750 from a treatment apparatus according to an embodiment. The measured spectra provides relative intensity values of first plurality of wavelength ranges and second plurality of wavelength values. The intensity values in the graph are relative and do not represent actual radiant powers due to non-linearity of spectrometer as function of wavelengths. In the figure all of the treatment apparatus light source(s) are configured to provide reference light for calibration purposes. Actual radiant powers used during the treatment would result to different relative peak intensity values. In general integral below each peak (integrated from first wavelength to a second wavelength such as from 270 nm to 280 nm) can be interpretant as radiant power which is associated to said range. Appropriate corrections to take in account energy dependency of wavelength of light should be taken in consideration as well (E=hc/lamda, wherein h is Planck's constant, c is speed of light and lamda is wavelength).

When the example treatment apparatus is configured to for treatment of a chronic wound of a first type following parameters and protocol are used in the treatment apparatus:

During the first period of time (of 60 seconds) following admirations of light are done (i.e. illuminating wound with a range of lights):
1) A first disinfecting wavelength range of light 760 (as measured ½ of max intensity value of the wavelength) from 260 nm to 280 nm. Peak maximum is 270 nm. Integral from 260 nm to 280 nm represents a first radiant power. Used radiant power for a treatment modality (of treatment of chronic wound of the first type) is 100 uW/cm2 (uW=microwatt)
2) A second disinfecting wavelength range of light 762 (as measured ½ of max intensity value of the wavelength) is from 305 nm to 320 nm. Peak maximum is 310 nm. Integral from 305 nm to 320 nm represents a second radiant power. Used radiant power for the treatment modality is 100 uW/cm2.
3) A third disinfecting wavelength range of light 764 (as measured ½ of max intensity value of the wavelength) is from 410 nm to 430 nm. Peak maximum is 420 nm. Integral from 410 nm to 430 nm represents a third radiant power. Used radiant power for the treatment modality is 3000 uW/cm2. This is administered during the first period of time.

According to embodiment the first, second and third disinfecting wavelength ranges are thus activated during the first period of time for 60 seconds when the apparatus is configured for treating the wound of the first type. After this first period of time a second period of time of 120 seconds is initiated immediately after the first period of time.
4) The first disinfecting wavelength range 760 is administered with radiant power of 100 uW/cm2.
5) In addition to the first disinfecting wavelength range 760, a fourth disinfecting wavelength range of light 766 is provided. The fourth disinfecting wavelength range 766 (as measured ½ of max intensity value of the wavelength) is from 635 nm to 645 nm as illustrated. Peak maximum is 630 nm. Integral from 635 nm to 645 nm represents a fourth radiant power. Used fourth radiant power for the treatment modality for the chronic wound of the first type is 500 uW/cm2.
6) A fifth disinfecting wavelength range of light 768 (as measured ½ of max intensity value of the wavelength) is from 825 nm to 860 nm as illustrated. Peak maximum is 850 nm. Integral from 825 nm to 860 nm represents a fifth radiant power. Used radiant power for the treatment modality is 1700 uW/cm2.

The treatment apparatus can be configured to execute above protocol of 1, 2, 3, 4, 5, 6 automatically or it can be controlled for example from a server system. Based on experiments the protocol has proven to be effective for the chronic wounds of the first type.

For the chronic would of the second type the same treatment apparatus can be configured to provide different profiles of light output namely for the chronic wound of the second type the treatment apparatus is programmed to follow following protocol

During a first period of time of 60 seconds (30-90 sec):
A) A first disinfecting wavelength range of light 760 (as measured ½ of max intensity value of the wavelength) from 260 nm to 280 nm. Peak maximum is 270 nm. Integral from 260 nm to 280 nm represents a first radiant power. Used radiant power for a treatment modality (of treatment of chronic wound of the second type) is 100 uW/cm2.
B) A fourth disinfecting wavelength range of light 766 is provided. The fourth disinfecting wavelength range 766 (as measured ½ of max intensity value of the wavelength) is from 635 nm to 645 nm as illustrated. Peak maximum is 630 nm. Integral from 635 m to 645 nm represents a fourth radiant power. Used fourth radiant power for the treatment modality for the chronic wound of the second type is 500 uW/cm2.
C) A fifth disinfecting wavelength range of light 768 (as measured ½ of max intensity value of the wavelength) is from 825 nm to 860 nm as illustrated. Peak maximum is 850 nm. Integral from 825 nm to 860 nm represents a fifth radiant power. Used radiant power for the treatment modality is 2700 uW/cm2.

After the first period of time a treatment sequence of duration of a second period of time (60 seconds) is followed right after.
D) A second disinfecting wavelength range of light 762 (as measured ½ of max intensity value of the wavelength) is from 305 nm to 320 nm. Peak maximum is 310 nm. Integral from 305 nm to 320 nm represents a second radiant power. Used radiant power for the treatment modality is 100 uW/cm2.
E) The fourth disinfecting wavelength range of light 766 is provided. Used fourth radiant power for the treatment modality for the chronic wound of the second type is 500 uW/cm2.
F) The fifth disinfecting wavelength range of light 768 is provided with radiant power of 2.5 mW/cm2.

After the second period of time a third period of time of treatment follows. Duration of the third period of time is preferably same as the second period of time. During the third period of time:
G) A sixth wavelength range is applied. The wavelength range for this is same as for the third wavelength in the example apparatus. Radiant power is 2 m W/cm2 and
H) The fourth disinfecting wavelength range is provided with power of 0.5 mW/cm2.

Indeed the treatment apparatus according to embodiment can be thus configured to provide treatment protocol for the chronic wound of the second type as well as the first type.

The aforementioned steps are only illustrative and other alternatives can also be provided where one or more steps are added, one or more steps are removed, or one or more steps are provided in a different sequence without departing from the scope of the claims.

1. A treatment apparatus for treatment of an open wound, the treatment apparatus comprising:
a processor, a light source and a distance measurement device, wherein the light source is arranged to emit light towards the open wound when in use;
wherein the distance measurement device is configured to measure the distance between the light source and the open wound and
wherein the processor is configured to control the light source to emit:
during a first period of time, a light comprising a first plurality of wavelength ranges, wherein at least one of the first plurality of wavelength ranges is a first disinfecting wavelength range of light having a first radiant power, the first disinfecting wavelength range of light is from 200 nm to 280 nm and the first radiant power is between 50 uW/cm² to 0.9 mW/cm² such that the first radiant power is adjusted based on this distance between the light source and the open wound to achieve the first radiant power that is required at the site of the wound; and
during a second period of time, a light comprising a second plurality of wavelength ranges, wherein at least one of the second plurality of wavelength ranges is the first disinfecting wavelength range having the first radiant power or a second disinfecting wavelength range of light having a second radiant power, the second disinfecting wavelength range of light is from 300 nm to 390 nm and the second radiant power is between 10 mW/cm² to 25 mW/cm²,
wherein the first time period is between 30 to 90 seconds, the second time period is between 30 to 90 seconds and wherein each respective radiant power is a radiant power of respective wavelength range of light at the wound.

2. The treatment apparatus according claim 1, wherein
the first plurality of wavelength ranges comprises the first disinfecting wavelength range of light having the first radiant power, the second disinfecting wavelength range of light having the second radiant power, and a third disinfecting wavelength range of light having a third radiant power; and
the second plurality of wavelength ranges comprises the first disinfecting range of wavelengths of light having the first radiant power, a fourth disinfecting range of wavelengths of light of a fourth radiant power and a fifth range of wavelengths of light from of a fifth radiant power.

3. The treatment apparatus according to claim 1, wherein
the first plurality wavelength ranges further includes a fourth disinfecting wavelength range of light having a fourth radiant power and a fifth wavelength range of light having a fifth radiant power; and
the second plurality of wavelength ranges further includes the fourth disinfecting range of wavelength of light of the fourth radiant power and the fifth range of wavelength of light from of the fifth radiant power.

4. The treatment apparatus according to claim 3, wherein the processor is further configured to control the light source to emit:
during a third period of time, a light comprising a third plurality of wavelength ranges, wherein the third plurality of wavelength ranges comprises a sixth range of wavelength of lights having a sixth radiant power, the fourth disinfecting range of wavelength of lights having the fourth radiant power and the fifth range of wavelength of lights having the fifth radiant power.

5. (canceled)

6. The treatment apparatus according to claim 2, wherein the third disinfecting wavelength range of light is from 400 nm to 430 nm and the third radiant power is between 3 mW/cm² to 200 mW/cm², wherein respective radiant power is a radiant power at the wound.

7. The treatment apparatus according to claim 2, wherein the fourth disinfecting wavelength range of light is from 610 nm to 660 nm and the fourth radiant power is between 0.5 mW/cm² to 200 mW/cm²; and
the fifth wavelength range of light is from 820 nm to 900 nm and the fifth radiant power is between 2 mW/cm² to 200 mW/cm², wherein each respective radiant power is a radiant power of respective wavelength range of light at the wound.

8. The treatment apparatus according to claim 4, wherein
the sixth wavelength range is from 400 to 430 nm and the sixth radiant power is between 2 mW/cm² to 200 mW/cm², wherein respective radiant power is a radiant power at the wound.

9. The treatment apparatus according to claim 1, wherein the third time period is between 30 to 90 seconds.

10. The treatment apparatus according to claim 1, wherein different ranges of wavelengths do not overlap with each other and have a wavelength range gap between each neighbouring ranges.

11. The treatment apparatus according to claim 3, wherein the fourth radiant power is provided as pulses having a frequency of 80 Hz to 120 Hz and with a duty ratio of ON: from 30:70 to 70:30.

12. The treatment apparatus according to claim 1, wherein the treatment apparatus is configured to control the light source to emit light when the wound type is a diabetes related wound.

13. (canceled)

14. A computer software product embodied on a non-transitory storage medium, wherein the computer software product is configured to run in a processor and is configured to cause the processor to control the light source according to claim 1.

15. A method of treatment of a chronic wound, wherein the wound is treated by providing during:
a first period of time of 30 to 90 seconds, a first plurality of wavelength ranges and radiant powers of:
a first disinfecting wavelength range from 200 nm to 280 nm and a first radiant power between 50 uW/cm² to 0.9 mW/cm²,
a second disinfecting wavelength range from 300 nm to 390 nm and a second radiant power is between 10 mW/cm² to 25 mW/cm², and
a third disinfecting wavelength range from 400 nm to 430 nm and a third radiant power is between 3 mW/cm² to 200 mW/cm²,
and
a second period of time of 60 to 180 seconds, a second plurality of wavelength ranges and radiant powers of:
the first disinfecting wavelength range from 200 nm to 280 nm and the first radiant power is between 50 uW/cm² to 0.9 mW/cm²,
a fourth disinfecting wavelength range from 610 nm to 660 nm and a fourth radiant power is between 0.5m W/cm² to 200 mW/cm², and
a fifth wavelength range from 820 nm to 900 nm and a fifth radiant power is between 2 mW/cm² to 200 mW/cm²,
wherein each respective radiant power is a radiant power of respective wavelength range of light at the wound.

16. A method of treatment of a chronic wound, wherein the wound is treated by providing during:
a first period of time of 30 to 90 seconds, a first plurality of wavelength ranges and radiant powers of:
a first disinfecting wavelength range is from 200 nm to 280 nm and a first radiant power is between 50 uW/cm² to 0.9 mW/cm²,
a third disinfecting wavelength range is from 610 nm to 660 nm and a fourth radiant power is between 0.5 mW/cm² to 200 mW/cm², and
a fourth wavelength range is from 820 nm to 900 nm and a fifth radiant power is between 2 mW/cm² to 200 mW/cm²,
a second period of time of 30 to 90 seconds, a second plurality of wavelength ranges and radiant powers of:
a second disinfecting wavelength range is from 300 nm to 390 nm and a second radiant power is between 10 mW/cm² to 25 mW/cm²,
the third disinfecting wavelength range is from 610 nm to 660 nm and the fourth radiant power is between 0.5 mW/cm² to 200 mW/cm², and
the fourth wavelength range is from 820 nm to 900 nm and the fifth radiant power is between 2 mW/cm² to 200 mW/cm²,
and
a third period of time of 30 to 90 seconds and a third plurality of wavelength ranges and radiant powers of:
a fifth wavelength range is from 400 to 430 nm and a sixth radiant power is between 2 mW/cm² to 200 mW/cm²,
the third disinfecting wavelength range is from 610 nm to 660 nm and the fourth radiant power is between 0.5 mW/cm² to 200 mW/cm², and
the fourth wavelength range is from 820 nm to 900 nm and the fifth radiant power is between 2 mW/cm² to 200 mW/cm²,
wherein respective radiant power is a radiant power at the wound.
